(19) United States
(12) Patent Application Publication
(10) Pub. No.: US 2021/0125705 A1
(43) Pub. Date: Apr. 29, 2021

(54) TECHNIQUES FOR DISPLAYING MEDICAL IMAGE DATA

(71) Applicant: Hyperfine Research, Inc,, Guilford, CT (US)

(72) Inventors: Brandon McGregor, Guilford, NY (US); Prantik Kundu, Guilford, CT (US); Arjun Sadanand, Brooklyn, NY (US); Scott Martin, Salem, CT (US)

(21) Appl. No.: 17/077,788

(22) Filed: Oct. 22, 2020

Related U.S. Application Data
(60) Provisional application No. 62/926,257, filed on Oct. 25, 2019.

(51) Int. Cl. G16H 30/20; G06F 16/955

(52) U.S. Cl. CPC G16H 30/20; G06F 16/955

(57) ABSTRACT

Described herein are techniques for displaying medical images on a first device by using information received from a second device different from the first device and at least one computer different from the first device and the second device. The method comprises: using at least one computer hardware processor of the first device to perform: obtaining, from the second device via at least one communication network, a uniform resource locator (URL) indicating a first plurality of parameters associated with a first view of medical image data for a subject; obtaining, from the at least one computer via the at least one communication network, the medical image data for the subject; after obtaining the URL and the medical image data for the subject: displaying a second view of the medical image data on the display of the first device using the first plurality of parameters indicated by the URL.

### CROSS-REFERENCE TO RELATED APPLICATIONS

This application claims priority under 35 U.S.C. § 119(e) to U.S. Provisional Application Ser. No. 62/926,257, filed Oct. 25, 2019, titled “DEEP STATE ROUTING SYSTEMS AND METHODS”, and which is incorporated by reference herein in its entirety.

### FIELD

The present disclosure relates generally to displaying medical images (e.g., magnetic resonance imaging (MRI) data) on devices and, more specifically, to techniques of configuring views of the medical images.

### BACKGROUND

Medical image data is obtained from performing diagnostic medical imaging on subjects (e.g., patients) to produce images of human body anatomy. For example, magnetic resonance imaging (MRI) involves detecting electromagnetic waves emitted in response to application of electromagnetic fields to the human body. The detected electromagnetic waves are used to produce images for use in diagnosing medical problems by medical care providers (e.g., physicians).

### SUMMARY

According to one aspect, a method for displaying medical images is provided. The method comprises: using at least one computer hardware processor of a first device to perform: obtaining, via at least one communication network, first medical image data for a subject; obtaining a first plurality of parameters associated with a first view of the first medical image data; displaying the first view of the first medical image data on a display of the first device using the first plurality of parameters; generating a first uniform resource locator (URL), the first URL indicating the first plurality of parameters; and transmitting the generated first URL to a second device for displaying second medical image data on the second device using the first plurality of parameters.

According to another aspect, a system for displaying medical images is provided. The system comprises: a display; at least one processor; and at least one non-transitory computer-readable storage medium storing instructions that, when executed by the at least one processor, cause the at least one processor to perform a method comprising: obtaining, via at least one communication network, first medical image data for a subject; obtaining a first plurality of parameters associated with a first view of the first medical image data; displaying the first view of the first medical image data on the display using the first plurality of parameters; generating a first uniform resource locator (URL), the first URL indicating the first plurality of parameters; and transmitting the generated first URL to a device, different from the system, for displaying second medical image data on the device using the first plurality of parameters.

According to another aspect, a non-transitory computer-readable storage medium is provided. The non-transitory computer-readable storage medium stores instructions that, when executed by at least one processor of a first device, cause the at least one processor to perform: obtaining, via at least one communication network, first medical image data for a subject; obtaining a first plurality of parameters associated with a first view of the first medical image data; displaying the first view of the first medical image data on a display of the first device using the first plurality of parameters; generating a first uniform resource locator (URL), the first URL indicating the first plurality of parameters; and transmitting the generated first URL to a second device for displaying second medical image data on the second device using the first plurality of parameters.

According to another aspect, described herein is a method for displaying medical images on a first device by using information received from a second device different from the first device and at least one computer different from the first device and the second device. The method comprises: using the first device to perform: obtaining, from the second device via at least one communication network, a uniform resource locator (URL) indicating a first plurality of parameters associated with a first view of medical image data for a subject; obtaining, from the at least one computer via the at least one communication network, at least some of the medical image data for the subject; after obtaining the URL and the at least some medical image data for the subject: displaying a second view of the at least some medical image data on a display of the first device using the first plurality of parameters indicated by the URL.

According another aspect, described herein is a system for displaying medical images by using information received from a device different from the system and at least one computer different from the system. The system comprises: a display; at least one processor; at least one non-transitory computer-readable storage medium storing instructions that, when executed by the at least one processor, cause the at least one processor to perform a method comprising: obtaining, from the device via at least one communication network, a uniform resource locator (URL) indicating a first plurality of parameters associated with a first view of medical image data for a subject; obtaining, from the at least one computer via the at least one communication network, at least some of the medical image data for the subject; after obtaining the URL and the at least some medical image data for the subject: displaying a second view of the at least some medical image data on the display using the first plurality of parameters indicated by the URL.

According to another aspect, a non-transitory computer-readable storage is provided. The non-transitory computer-readable storage medium stores instructions that, when executed by at least one processor of a first device, cause the at least one processor to perform: obtaining, from a second device different from the first device via at least one communication network, a uniform resource locator (URL) indicating a first plurality of parameters associated with a first view of medical image data for a subject; obtaining, from at least one computer different from the first device via the at least one communication network, at least some of the medical image data for a subject; and after obtaining the URL and the medical image data for the subject: displaying a second view of the at least some medical image data on a display of the first device using the first plurality of parameters indicated by the URL.

### BRIEF DESCRIPTION OF THE DRAWINGS

Various aspects and embodiments will be described with reference to the following figures.

It should be appreciated that the figures are not necessarily drawn to scale.

FIG. 1 shows an environment in which aspects of the technology described herein may be implemented.

FIG. 2 is a flowchart of a process for displaying medical images, according to some embodiments of the technology described herein.

FIG. 3 is a flowchart of a process for displaying medical images on a device using information received from a different device, according to some embodiments of the technology described herein.

FIG. 4 is a flowchart of a process for displaying a previously displayed view of medical image data, according to some embodiments of the technology described herein.

FIG. 5 is a flowchart of a process for sharing a view of medical image data, according to some embodiments of the technology described herein.

FIG. 6A shows a file storing parameters associated with a view of medical image data, according to some embodiments of the technology described herein.

FIG. 6B illustrates an example generated URL indicating the parameters shown in FIG. 6A, according to some embodiments of the technology described herein.

FIG. 7A illustrates an example of a first view displayed on a first device and a first URL indicating parameters associated with the first view, according to some embodiments of the technology described herein.

FIG. 7B illustrates an example of a second view obtained from modifying the first view and a second URL indicating parameters associated with the second view, according to some embodiments of the technology described herein.

FIG. 7C illustrates a screenshot of a user input of a graphical user interface (GUI) element for sharing the second view of FIG. 7B, according to some embodiments of the technology described herein.

FIG. 7D illustrates an example of a third view displayed on a second device using the second URL of FIG. 7B, according to some embodiments of the technology described herein.

FIG. 8 shows an illustrative computer on which aspects of the technology of the described herein may be implemented.

### DETAILED DESCRIPTION

Medical care providers (e.g., clinicians) often collaborate to provide medical care for a subject (e.g., a patient). In many instances, the collaboration may involve medical care providers using different computing devices to view and examine medical image data. For example, a first clinician analyzing a three-dimensional (3D) view of a computerized tomography (CT) scan on a first device may wish to share the 3D view of the CT scan with a second clinician (e.g., in order to confirm a diagnosis).

The inventors have recognized that conventional techniques of displaying medical image data do not allow for efficient collaboration across multiple devices. When users of different devices need to examine a view of medical image data, conventional techniques involve manually recreating a view on each device. For example, a first clinician obtains a view of medical image data (e.g., MRI scan data) on a first device by searching for the data (e.g., in an electronic medical record (EMR) system), browsing and selecting a scan from the data, and then manually adjusting visual properties of the displayed scan to obtain the view. The first clinician may wish to involve one or more clinicians in analyzing the medical image data. For example, the first clinician may consult the other clinician(s) on a diagnosis determined from the view of the medical image data. The other clinician(s) may be using one or more devices different from the first device. To obtain the view obtained by the first clinician, the other clinician(s) would have to go through the time-consuming process of searching for the medical image data from the dataset, browsing and selecting the scan from the data, and then adjusting a view on the (e.g., by rotating, zooming, panning, modifying brightness, and/or modifying contrast) device(s). Moreover, the view obtained by the other clinician(s) may not be an accurate replication of the view obtained by the first clinician. Thus, conventional techniques for displaying medical images add significant time and effort required to provide medical care (e.g., when clinicians need to collaborate regarding a view of medical image data).

Accordingly, the inventors have developed techniques of displaying medical images that allow medical imaging systems to efficiently replicate a graphical user interface (GUI) state across multiple devices. In particular, the techniques developed by the inventors involve generating a uniform record locator (URL) indicating parameters associated with a medical data view shown in a GUI displayed on a first device. To replicate that same view on a second device, the URL indicating the parameters may be transmitted to the second device. When accessed by an Internet browser executing on the second device, the URL causes the second device to replicate the view of the medical image data from the first device using the parameters indicated by the URL. This allows clinicians to efficiently share GUI states (e.g., views of medical image data among different devices). For example, a first clinician using a first device may share a view of medical image data (e.g., a view of an MRI scan) with a second clinician on a second device by simply sharing a URL. Accordingly, techniques described herein reduce the number of computations that are performed by the second device to display the view on the second device.

The inventors have further recognized that conventional medical imaging technology is inefficient. In particular, GUIs utilized in conventional medical imaging technology are inefficient because they do not efficiently store GUI states. For example, a software application that provides views of medical image data (e.g., MRI scans) may not preserve GUI states (e.g., which include views of medical image data) generated by the software application. Thus, if a user wants to access one or more previously displayed views, the user may have to repeat steps taken to obtain the views. For example, when the user wants to obtain the views at a later time, the user may be required to take steps to access the medical image data, and manually adjust a default view of the medical image data in a GUI to obtain the previously displayed views. Accordingly, GUIs provided by conventional medical image technology are (1) unable to efficiently store and navigate user interface states; and (2) cumbersome for users to use in the context of collaboration and returning to previous user interface states.

Accordingly, the inventors have developed techniques that improve medical imaging technology. In particular, techniques described herein improve GUIs utilized in medical imaging technology. Techniques described herein provide medical imaging technology that stores user interface states of a software application (e.g., for viewing medical image data as URLs). For example, a system may save a view of MRI scan data by saving a URL indicating parameters associated with the view. When the user wants to access the view of the medical image data later, the user may simply access a URL associated with the view. The system may regenerate the view using the parameters indicated by the URL. Accordingly, medical imaging techniques described herein provide a graphical user interface (GUI) that efficiently navigates among multiple different states. The techniques reduce the number of computations required to access the views. For example, techniques described herein may provide a GUI that efficiently navigates among multiple different views of medical image data, and eliminates computations that would be required by conventional medical image technology to navigate among the views of medical image data and/or recreate previously generated views.

Some embodiments described herein address all the above-described issues that the inventors have recognized with medical imaging technology. However, it should be appreciated that not every embodiment described herein addresses every one of these issues. It should also be appreciated that embodiments of the technology described herein may be used for purposes other than addressing the above-discussed issues of conventional techniques of medical imaging.

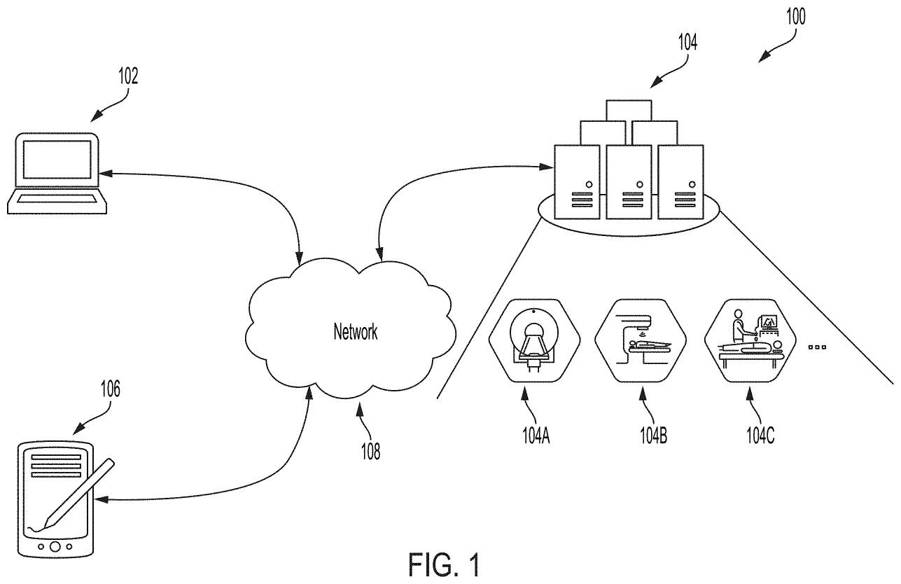

FIG. 1 shows an environment 100 in which aspects of the technology described herein may be implemented. Environment 100 includes a first user device 102, a medical image data server 104, a second user device 106, and a communication network 108.

The first user device 102 may be any computing device. In the example of FIG. 1, the first user device 102 is a laptop. In some embodiments, the first user device 102 may be a mobile device such as a smartphone, a tablet, or another mobile computing device. In some embodiments, the first user device 102 may be a desktop computer or a workstation. For example, the first user device 102 may be a shared workstation at a hospital for use by clinicians to view medical image data.

In some embodiments, the user device 102 may be coupled to a display on which views of medical image data may be shown. In some embodiments, the display may be integrated with the user device 102. In the example of FIG. 1, the display is a screen of a laptop. In another example, the display may be a screen of a mobile device (e.g., a smartphone or tablet). In some embodiments, the display may be an external display (e.g., a monitor, or television) in communication with the user device 102.

In some embodiments, the user device 102 may include a network interface for communicating with one or more other devices (e.g., through communication network 108). For example, the user device 102 may include a network interface device (e.g., a network interface card) that the user device 102 uses to communicate through the network 108. The user device 102 may be configured to use the network interface device to communicate with other devices. For example, the user device 102 may use the network interface device to communicate with medical image data server 104 (e.g., to obtain medical image data) and/or user device 106 (e.g., to share a view of medical image data).

In some embodiments, the user device 102 may be configured to execute a software application for viewing medical image data. The software application may be configured to render a display of medical image data (e.g., obtained from the server 104) on a display of the user device 102. In some embodiments, the software application may be a program written using a scripting language. The user device 102 may execute the program to render the display of medical image data. For example, the software application may be a JAVASCRIPT program. In this example, the computing device 102 may execute the program using an Internet browser application to render images from medical image data on a display of the user device 102. In another example, the software application may be hypertext markup language (HTML) and/or cascading style sheets (CSS) code. In another example, the software application may include a combination of JAVASCRIPT code, HTML code, and/or CSS code. In another example, the software application may be a program written using PYTHON, C++, JAVA, or another programming language.

In some embodiments, the user device 102 may be configured to generate views of medical image data to be shown on a display of the user device 102. The user device 102 may be configured to: (1) obtain medical image data for a subject; and (2) display a view of the medical image data on a display of the user device 102. The user device 102 may be configured to provide a user interface for displaying views of the medical image data. The user interface may be an interactive user interface through which a user can create, modify, and/or store views of medical image data. The user device 102 may be configured to generate a view using user input provided through the user interface. The user device 102 may be configured to generate the view by: (1) obtaining user input (e.g., click, drag, and/or scroll) through the user interface; and (2) setting visual properties of the view according to the user input. In some embodiments, the user device 102 may be configured to allow the user to modify a first view of medical image data to obtain a second view of the medical image data. For example, the user interface may allow the user to modify the first view by rotating, panning, zooming, adjusting brightness, adjusting contrast, or performing other modifications to obtain the second view. In one example, the user interface may allow the user to use a mouse and/or any other type of input device to modify a view shown on the display (e.g., by clicking and dragging). In another example, the user interface may provide a touch screen interface through which a user can modify a view (e.g., by tapping, dragging, and/or pinching). In another example, the user interface may be an extended reality (e.g., augmented reality or virtual reality) interface in which the user can modify a view of medical image data.

In some embodiments, a view of medical image data may be associated with one or more parameters. In some embodiments, the parameter(s) may indicate visual properties of the view. In some embodiments, the parameter(s) may indicate one or more of the following:

An identifier of the medical image data. The identifier of the medical image data may include an identifier of one or more medical image scans. For example, the parameters may indicate, for each of the medical image scan(s), an alphanumeric string identifying the medical image scan.

A position of a view plane along one or more axes. For example, the parameters may indicate a position of the view plane along an x-axis, y-axis, and/or z-axis (e.g., of a displayed volume). In some embodiments, the position of the view plane along a respective axis may be indicated by a value from a range of values (e.g., between −1 and 1, −10 to 10, −100 to 100, −1000 to 1000, etc.).

An extent of a pan from a point on one or more axes. For example, the parameters may indicate an extent of a pan from the origin along the x-axis and y-axis of the view. In some embodiments, the pan may be indicated by a value from a range of values (e.g., between −1 and 1, −10 to 10, −100 to 100, −1000 to 1000).

A rotation of a volume in the view about an axis of a coordinate system. In some embodiments, the indication of rotation may be a rotation matrix of values representing rotation of the volume about an x, y, or z axis of the coordinate system. For example, the indication of rotation may be a 3×3 rotation matrix that rotates the volume. In some embodiments, each value in the rotation matrix may be a value from a range of values (e.g., between −1 and 1, −10 to 10, −100 to 100, −1000 to 1000).

A view mode for the view of the medical image data. In some embodiments, a view mode may be one of a 2D view or a 3D view. In some embodiments, the view mode may be a multiplanar reconstruction (MPR) view. An MPR view comprises a view obtained from converting medical image data from one plane into another plane. In some embodiments, the indication of the view mode may be a value (e.g., a string, or an integer) specifying the view mode.

A brightness of the view of the medical image data. In some embodiments, the indication of the brightness may be a value from a range of values (e.g., between −1 and 1, −10 to 10, −100 to 100, −1000 to 1000).

A contrast of the view of the medical image data. In some embodiments, the indication of the contrast may be a value from a range of values (e.g., between −1 and 1, −10 to 10, −100 to 100, −1000 to 1000).

A zoom value indicating a degree to which the view is zoomed in. In some embodiments, the indication of the zoom may be a value from a range of values (e.g., between −1 and 1, −10 to 10, −100 to 100, −1000 to 1000).

A clipping plane. For example, the indication may be a matrix including three values defining the clipping plane. The values may indicate points along axes (e.g., [“x”:0.967, “y”: −0.16, “z”: −0.19]). In some embodiments, the indication may include a clip offset. The clip offset may indicate an offset of a viewing plane from the clipping plane. For example, a matrix of values defining the clipping plane may include a clip offset value with three values defining the clipping plane (e.g., [“x”:0.967, “y”: −0.16, “z”: −0.19, “clipOffset”: 0.2]).

An interpolation toggle value. The interpolation toggle may be a set on or off (e.g., using a Boolean or integer value). The system may be configured to automatically interpolate an image of the medical image data to a size of a display when the toggle is set on. For example, the system may automatically interpolate a 512×512 pixel image to a 1024×1024 pixel display.

In some embodiments, a view of the medical image data may include one or more panes. Each pane may display respective medical image data. In some embodiments, each of the pane(s) may be a respective window of the view of the medical image data. For example, a first pane of the view may be a first window displaying a first medical scan, and a second pane of the view may be a second window displaying a second medical scan different from the first medical scan. In another example, a first pane may display a first view of the medical image data, and a second pane may display a second view of the medical image data. In some embodiments, parameters associated with a view of medical image data may include parameters for each pane of the view. For example, the parameters may include (1) a first set of parameters indicating visual properties of a first pane of the view, and (2) a second set of parameters indicating visual properties of a second plane of the view. Example parameters are described herein. In some embodiments, parameters for a pane may be named to identify the association with the pane. For example, brightness for a first pane may be indicated by a variable “bri1” and brightness for a second pane may be indicated by a variable “bri2”. In another example, zoom for a first pane may be indicated by a variable “zoom1” and zoom for a second pane may be indicated by a variable “zoom2”. In some embodiments, the parameters may include global parameters indicating view properties of all the panes. For example, the global parameters may include a global zoom parameter indicating a zoom for all the panes.

In some embodiments, the system may be configured to determine values for the parameters according to adjustments to a view shown on a display of the user device 102. For example, the user device 102 may display a first view associated with a first set of parameters. The first view may be modified by a user (e.g., by zooming, rotating, and/or modifying brightness) to obtain a second view. The user device 102 may be configured to adjust the first set of parameters in response to one or more modifications by the user. For example, the system may modify a brightness parameter in the first set of parameters when a user modifies a brightness of the first view and modify a zoom parameter when the user modifies a zoom of the first view. Thus, the second view may be associated with a second set of parameters different from the first set of parameters.

In some embodiments, the user device 102 may be configured to provide a default view of medical image data. For example, the user device 102 may store a default set of parameters which the user device 102 uses for generating a default view of medical image data. In some embodiments, the user device 102 may be configured to store default sets of parameters for different types of medical image data. For example, the system may store a first default set of parameters for a default view of MRI scan data, and a second default set of parameters for a default view of CT scan data. The system may use the default view when no other view has been specified (e.g., by a user of the device). The system may be configured to generate views different from a default view using user input (e.g., through a user interface for modifying a view of medical image data).

In some embodiments, the user device 102 may be configured to save a view (e.g., created by a user). The user device 102 may be configured to preserve the view by storing parameters associated with the view. In some embodiments, the user device 102 may be configured to generate a uniform resource locator (URL) indicating the parameters associated with the view. The parameters may be alphanumeric values indicated by the URL. In some embodiments, the user device 102 may be configured to store a URL indicating the parameters. For example, the system may store the URL indicating the parameters associated with the view in a datastore (e.g., a hard drive) of the user device 102. In another example, the system may store the URL in a cloud storage system through the network 108.

In some embodiments, the user device 102 may be configured to determine a view using a previously stored URL indicating parameters associated with the view. The system may access the URL from memory, and use the URL to render a display of the view on the user device 102. The user device 102 may be configured to use parameters indicated by the URL as input values in a software application (e.g., a JAVASCRIPT program) for rendering views of medical image data on a display of the user device 102. For example, the user device 102 may provide parameter values as inputs to one or more functions of the software application that use the inputs to generate the view of the medical image data. The view shown on the display of the user device 102 may have visual properties indicated by the URL.

In some embodiments, the user device 102 may be configured to share a view of medical image data determined by the user device 102 with one or more other devices (e.g., user device 106). For example, a first clinician using the user device 102 may share a view of an MRI scan created by the first clinician on the user device 102 with one or more other clinicians using one or more devices different from the user device 102. The user device 102 may be configured to obtain a copy of the medical image data from a data store (e.g., from medical image data server 104). The copy of the medical image data obtained by the user device 102 may be referred to as “first medical image data.” The user device 102 may be configured to use the first medical image data to generate the view of the medical image data on a display of the user device 102 (e.g., using user input to set the view). The user device 102 may be configured to share the view with the other device(s) by: (1) generating a URL indicating parameters associated with the view; and (2) transmitting the generated URL to the device(s). The URL, when accessed by the device(s), may cause the device(s) to obtain a copy of the medical image data. The copy of the medical image data obtained by the device(s) may be referred to as “second medical image data.” The second device may use the parameters indicated by the URL to determine a view of the medical image data on the second device. The parameters indicated by the URL may allow the device(s) to replicate the view from the user device 102. For example, the device(s) may provide the parameters indicated by the URL as input to a computer program (e.g., a JAVASCRIPT program) for rendering the view on a display of the second device. By using the parameters indicated by the URL, the device(s) may display a view that is associated with the same parameters as the view on the user device 102.

In some embodiments, the user device 102 may be configured to share a view with one or more devices different from the user device 102 in response to a user making one or more selections in a graphical user interface (GUI). The user device 102 may be configured to generate a selectable GUI element in a user interface through which a user may initiate sharing of the view with the second device. After receiving the user selection of the GUI element, the user device 102 may prompt the user to provide input specifying one or more recipients. For example, the user device 102 may prompt the user for an email address, a username, name, or other identification of a recipient. After receiving the input specifying the recipient(s), the user device 102 may transmit a URL indicating parameters associated with the view to the recipient(s) (e.g., to user device 106). For example, in response to receiving input indicating a user command to send, the user device 102 may transmit the URL indicating parameters associated with the view to the device(s).

In some embodiments, the medical image data server 104 of FIG. 1 may be configured to store medical image data. The medical image data server 104 may include storage hardware. For example, the storage hardware may include one or more solid state hard drives (SSDs), hard disk drives (HDD), or other type of storage hardware for storing the medical image data. The medical image data server 104 may include a database management system (DBMS) that manages data stored by the medical image data server 104. For example, the DBMS may allocate data among hardware storage resources, archive data, backup data, and perform other tasks. The DBMS may provide an interface through which other computing devices (e.g., user device 102 and user device 106) can interact with the medical image data server 104. Computing devices may access data from the medical image data server 104 through the database management system. For example, a computing device may transmit a request to the DBMS of the medical image data server 104 to read, store, modify, and/or delete data from the medical image data server 104.

In some embodiments, the medical image data server 104 may be configured to store medical image data obtained from performing medical imaging. For example, the medical image data server 104 may store magnetic resonance imaging (MRI) data 104A, X-Ray data 104B, CT Scan data 104C, and other types of medical image data. The medical image server 104 may be configured to store the medical image data in image files. For example, the medical image server 104 may store image files in ANALYZE format, NIFTI format, MINC format, DICOM format, and/or another image format.

In some embodiments, the medical image data server 104 may be a cloud-based server. The cloud-based server may be configured to provide data storage to one or more computer systems. For example, the cloud-based server may provide data storage to a medical institution (e.g., a hospital network). In some embodiments, the medical image data server 104 may be a component of an electronic medical record (EMR) system. For example, the medical image data server 104 may form a portion of data stored in the EMR system.

In some embodiments, a URL indicating parameters associated with a view may indicate data stored in the medical image data server 104 to be used for generating the view. The URL may indicate particular set of data from which the view is generated. In some embodiments, the URL may indicate one or more files (e.g., storing medical image data) from which a view is generated. For example, the URL may include an alphanumeric identifier for the file(s) in the URL. A computing device may use the indication of the file(s) to access the file(s) from the medical image data server 104. For example, a computing device (e.g., user device 106) may (1) use an indication of the URL to generate a request; and (2) transmit the request to the medical image data server 104. The medical image data server 104 may provide medical image data identified in the request to the computing device when the server 104 receives the request.

In some embodiments, the second user device 106 of FIG. 1 may be any suitable type of computing device. In the example of FIG. 1, the second user device 106 is a smartphone. In some embodiments, the second user device 106 may be a laptop computer, a tablet, or another mobile device. In some embodiments, the second user device 106 may be a desktop computer or a workstation. For example, the second user device 106 may be a shared workstation at a hospital for use by clinicians to view medical image data.

In some embodiments, the user device 106 may include a display on which images of medical image data may be shown. In some embodiments, the display may be integrated with the user device 106. In the example of FIG. 1, the display is a screen of a smartphone. In another example, the display may be a screen of a laptop computer. In some embodiments, the display may be an external display (e.g., a monitor, or television) in communication with the user device 106.

In some embodiments, the user device 106 may include a network interface for communicating with one or more other devices through the communication network 108 (e.g., the Internet). For example, the user device 106 may include a network interface device (e.g., a network interface card) that the user device 106 uses to communicate through the network 108. The user device 106 may be configured to use the network interface device to communicate (e.g., over network 108) with other systems (e.g., user device 102 and/or medical image data server 104).

In some embodiments, the user device 106 may include a software application for viewing medical image data. The software application may be configured to render a display of medical image data (e.g., obtained from the server 104). For example, the software application may be a JAVASCRIPT program that the user device 106 executes using an Internet browser application to render views of medical image data. Example software applications for viewing medical image data are described herein.

In some embodiments, the second user device 106 of FIG. 1 may be configured to generate views of medical image data to be shown on a display of the user device 106. The user device 106 may be configured to: (1) obtain medical image data for a subject; and (2) obtain parameters associated with a view of the medical image data to be shown on a display of the user device 106. The user device 106 may be configured to provide a user interface for displaying views of the medical image data. The user interface may be an interactive user interface through which a user can create views of medical image data. The user device 106 may be configured to obtain the parameters associated with the view by determining the parameters using user input provided through the user interface. The user interface may allow the user to modify a first view of medical image data to obtain a second view of the medical image data. For example, the user interface may allow the user to modify the first view by rotating, panning, zooming, or performing another modification to obtain the second view. In one example, the user interface may allow the user to use a mouse or other input device to modify a view shown on the display (e.g., by clicking and dragging). In another example, the user interface may provide a touch screen interface through which a user can modify a view (e.g., by tapping, dragging, and/or pinching). In another example, the user interface may be an extended reality (e.g., augmented reality or virtual reality) interface in which the user can modify a view of medical image data.

In some embodiments, the user device 106 may be configured to display a view of medical image data (e.g., on a display of the device 106) by (1) obtaining, from another device (e.g., user device 102), a URL indicating parameters associated with a first view of the medical image data (e.g., displayed on the user device 102); (2) obtaining the medical image data (e.g., from medical image data server 104); and (3) displaying a second view of the medical image data on a display of the device 106 using parameters indicated by the URL obtained from the other device. The user device 106 may replicate the first view shown on the other device on a display of the user device 106 (e.g., to allow a user of the user device 106 to collaborate with a user of the user device 102). The second view displayed by the user device 106 may be a replication of the view displayed by the user device 102. For example, the second view of the medical image data shown on the display of the user device 106 may have the same visual properties (e.g., zoom percentage, rotation angle, brightness, contrast, and/or other visual properties) as a view determined by user device 102. Example view properties are described herein.

In some embodiments, the first view and the second view of the medical image data may be substantially the same. Two views are substantially the same when all values of parameters associated with the first view are within 5% of values of parameters associated with the second view. For example, a first view may be substantially the same as a second view when values for brightness, contrast, zoom, and rotation angle associated with the first view are within 5% of values for brightness, contrast, zoom, and rotation angle associated with the second view.

In some embodiments, the user device 106 may be configured to display medical image data using a URL obtained from another device (e.g., user device 102) by using parameters indicated by the URL. The user device 106 may be configured to use the parameters in a software application to render the display of the view. For example, the user device 106 may use the parameters in a JAVASCRIPT program that, when executed, renders a view from the medical image data on the display of the device 106. The JAVASCRIPT program may use the parameters indicated by the URL (e.g., as inputs to function(s) of the JAVASCRIPT program and/or to define variables in the JAVASCRIPT program). The user device 106 may be configured to use an Internet browser application to execute the JAVASCRIPT program.

In some embodiments, the user device 106 may be configured to obtain the medical image data (e.g., from medical image data server 104) in response to obtaining a URL (e.g., from user device 102). The user device 106 may be configured to obtain a copy of the medical image data using the URL. For example, the URL may indicate an identifier of the medical image data (e.g., one or more files). The user device 106 may be configured to request the identified medical image data from the medical image data server 104 (e.g., by submitting a database request). The user device 106 may obtain a copy of the medical image data in response to transmitting a request to the medical image data server 104.

Although the example embodiment of FIG. 1 shows a single user device 106 that obtains a URL from the user device 102, in some embodiments multiple devices may obtain the URL form the user device 102. Each of the multiple user devices may be configured to display a view of medical image data as described herein with reference to user device 106. For example, a user of user device 102 may wish to present a view of medical image data to multiple users using multiple different devices. The user device 102 may be configured to transmit a URL indicating parameters associated with a view displayed on user device 102 to the different devices. Each of the devices may use the URL transmitted from the user device 102 to display a view of the medical image data using the parameters indicated by the URL.

In some embodiments, the communication network 108 of FIG. 1 may be any network through which the user device 102, user device 106, and the medical image data server 104 can communicate. In some embodiments, the network 108 may be the Internet, a local area network (LAN), a wide area network (WAN), a cellular network, an ad hoc network, and/or any other suitable type of network, as aspects of the technology described herein are not limited in this respect. In some embodiments, the network 108 may include one or more wired links, one or more wireless links, and/or any suitable combination thereof. For example, the network 108 may be an internal communication network of a hospital.

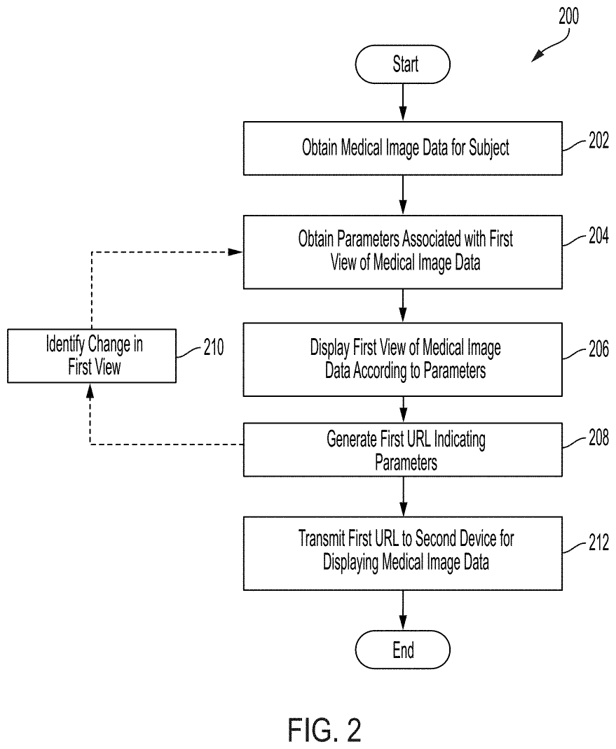

FIG. 2 is a flowchart of an illustrative process 200 for displaying medical images, according to some embodiments of the technology described herein. Process 200 may be performed by any suitable computing device. For example, process 200 may be performed by user device 102 described herein with reference to FIG. 1.

Process 200 starts at block 202, where the system performing process 200 obtains medical image data for a subject. In some embodiments, the system may be configured to obtain the medical image data from a source external to the system (e.g., medical image data server 104). For example, the system may: (1) transmit, to a server, a request indicating a request for the medical image data for the subject; and (2) receive the medical image data in response to the request. In some embodiments, the system may be configured to obtain the medical image data for the subject in response to user input obtained through a user interface. The system may receive a request to view the medical image data for the subject through a GUI provided by the system. For example, the system may provide an Internet website through which the user may request to view medical image data. The system may obtain the medical image data in response to receiving a request to view the medical image data.

In some embodiments, the medical image data may be stored locally on the system. The system may include local storage (e.g., a hard drive or any other suitable type of memory) in which the medical image data for the subject is stored. The system may retrieve the medical image data from the local storage. For example, the system may have previously obtained the medical image data from an external server, and stored the medical image data in local storage (e.g., for faster access). At block 202, the system may obtain the medical image data from the local storage.

In some embodiments, the medical image data may be of any suitable type. For example, the medical image data may be MRI data, ultrasound scan data, x-ray scan data, CT scan data, PET scan data, or other type of medical imaging scan. Examples of medical image data are described herein. In some embodiments, the medical image data may include one or more image files storing the medical image data. The system may be configured to obtain the image file(s) storing the medical image data. For example, the system may transmit, to a medical image data server, a request indicating an identifier for one or more scans. The system may receive the scan(s) from the server in response to the request.

After obtaining the medical image data, process 200 proceeds to block 204, where the system obtains parameters associated with a first view of the medical image data. The parameters may indicate visual properties of the first view of the medical image data. Example parameters are described herein.

In some embodiments, the system may be configured to obtain the parameters associated with the first view as user input. The user input may indicate values of the parameters. For example, the system may receive textual input indicating the values of the parameters. In some embodiments, the system may be configured to obtain the parameters by determining the parameters from user input. The user may make modifications to another view in a graphical user interface (GUI) (e.g., by clicking, tapping, dragging, and/or making other adjustments) to obtain the first view. The system may determine the parameters by: (1) determining the modifications; and (2) determining the parameters according to the modifications. For example, the system may detect user input (e.g., click and drag) to rotate a volume displayed in the GUI. In this example, the system may determine parameters indicating rotation according to the user input.

In some embodiments, the system may be configured to obtain the parameters associated with the first view by obtaining default parameters. For example, the system may obtain the parameters by setting the parameters to default values (e.g., stored by the system). In some embodiments, the system may be configured to obtain the parameters associated with the first view from a URL. For example, the system may use a previously generated URL indicating the parameters associated with the first view. In this example, the system may obtain the parameters from the URL. The system may use the parameters indicated by the URL to display the first view of the medical image data (e.g., as described below at block 206).

Next, process 200 proceeds to block 206, where the system displays the first view of the medical image data according to the parameters. In some embodiments, the parameters may be used as variable values in a software application for rendering the first view of the medical image data. For example, the parameters may be used as input values in one or more functions of the software application. In some embodiments, the software application may be JAVASCRIPT program. The system may execute the JAVASCRIPT program (e.g., using an Internet browser application) to render the display of the first view of the medical image data. The JAVASCRIPT program may use the parameters associated with the first view to render the display. In some embodiments, the system may be configured to render the display of the first view of the medical image data using ray tracing.

In some embodiments, the system may be configured to display the first view of the medical image data on a display of the system. For example, the system may display the first view on a display monitor of the system. In another example, the system may display the first view on a touchscreen display of the system. In some embodiments, the system may be configured to display the first view in a graphical user interface (GUI) through which a user may interact with the first view (e.g., my modifying the first view). In some embodiments, the GUI may be an Internet website displayed by the system using an Internet browser application.

In some embodiments, the first view may include one or more panes. Each of the pane(s) may display a medical image scan. The parameters associated with the first view may indicate visual properties for each of the pane(s). For example, the first view may include two different MRI scans. Each of the two scans may be associated with a respective brightness, contrast, and zoom level. In some embodiments, the parameters may include a set of parameters for each of the medical image scan(s) of the first view. Continuing with the example of the two MRI scans, the parameters may include (1) a first set of parameters indicating brightness, contrast, and zoom level for the first MRI scan; and (2) a second set of parameters indicating brightness, contrast, and zoom level for the second MRI scan.

Next, process 200 proceeds to block 208, where the system generates a first uniform record locator (URL) indicating the parameters associated with the first view. The system may be configured to encode the parameters associated with the first view in the URL by encoding, for each of the parameters, a parameter identifier and an associated value. In some embodiments, the system may be configured to generate the URL by obtaining parameters from a software application (e.g., for displaying views of the medical image data). For example, the system may obtain the parameters from a file (e.g., a JSON file) used by the software application to generate a display of the first view.

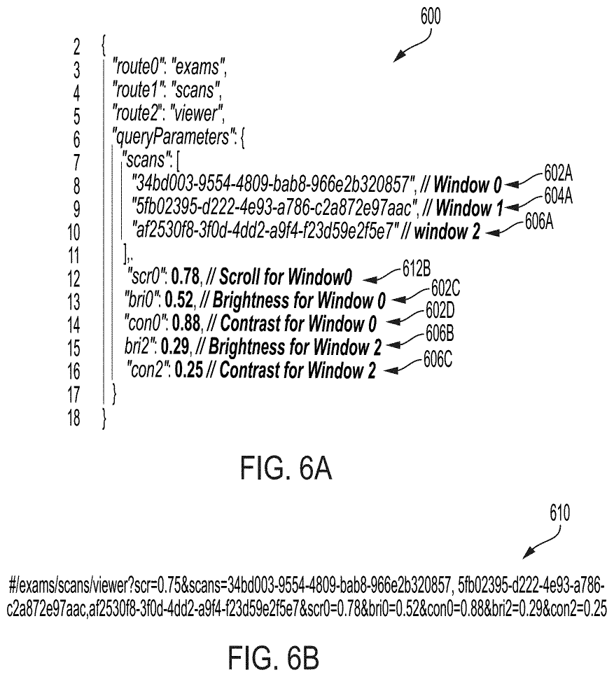

FIG. 6A shows a file 600 storing the parameters associated with a view of medical image data (e.g., used by a software application for displaying the view). The file 600 includes an identifier 602A for a first scan of the view, an identifier 604A for a second scan of the view, and an identifier 606A for a third scan of the view. The file 600 includes, for the first scan: (1) a scroll setting 602B of 0.78; (2) a brightness 602C of 0.52; and (3) a contrast 602D of 0.88. The file 600 includes, for the third scan: (1) a brightness 606B of 0.29; and (2) a contrast 606C of 0.25

FIG. 6B illustrates an example generated URL 610 indicating the parameters stored in the file 600, according to some embodiments. For example, the URL 600 indicates the identifiers 602A, 604A, 606A of the scans in the first view. The URL 600 indicates, for the first scan: (1) a scroll setting of 0.78; (2) a brightness of 0.52; and (3) a contrast of 0.88. The URL 600 indicates, for the third scan: (1) a brightness of 0.29; and (2) a contrast of 0.25. As shown in the example of FIGS. 6A-B, the parameters indicated by the URL 600 correspond to the parameters in the software code 610.

Returning to process 200, as indicated by the dotted line, process 200 may proceed to block 210 when there is a change in the first view. However, if there is no change in the first view, then process 200 proceeds to block 212.

At block 210, the system identifies a changes the first view of the medical image data. For example, a user viewing the first view of the medical image data may adjust visual properties (e.g., zoom, brightness, contrast, and/or other properties) of the first view. The system may identify the change the first view in response to user inputs for adjusting the visual properties. The change in the first view may result in changes to one or more of the parameters associated with the first view. The system may be configured to change the parameter(s) in response to the user modifications in the first view. For example, a user may make modifications in the first view of the medical image data in response to user input. The system may be configured to change the URL to indicate the updated parameter(s) in response to the change in the first view.

In some embodiments, the system may be configured to determine a change in a URL storing parameter(s) associated with the first view. For example, the URL may be changed by a user modification of the URL and/or programmatic modification of the URL. The system may use updated parameter(s) indicated by the changed URL to update the first view. In some embodiments, the system may be configured to use a software event listener to monitor for changes in the URL. For example, the listener may be a window listener that detects changes in a URL in a URL field of an Internet browser application. The listener may indicate when there is a change in parameter(s) indicated by the URL. The system may be configured to update the first view when the listener indicates the change in the parameter(s). The system may execute an application (e.g., JAVASCRIPT code) to use the changed parameter(s) to render the first view using the changed parameter(s). For example, the system may parse the changed parameter(s) into a JAVASCRIPT object that is used by a JAVASCRIPT application to render the first view using the changed parameter(s).

In some embodiments, the system may be configured to automatically apply changes to the parameter(s) associated with the first view. For example, the system may include programmatic display enhancement software that, when executed, applies changes to the parameter(s) associated with the first view. After identifying the changed view at block 210, the process 200 returns to block 204.

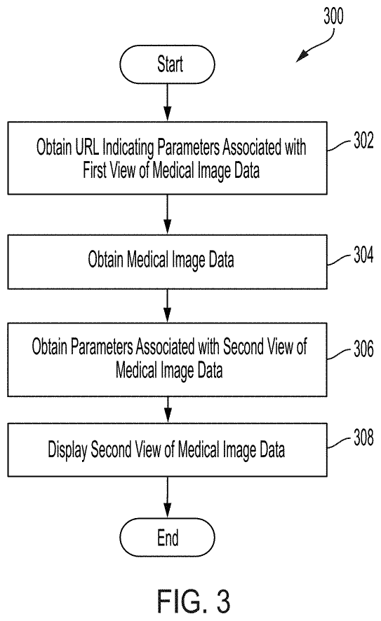

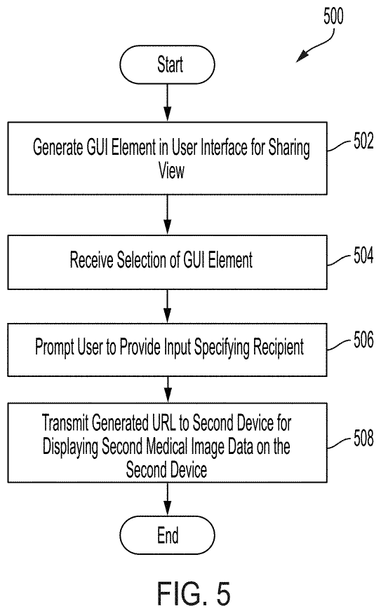

At block 212, the system transmits the generated URL to a second device (e.g. user device 106) for displaying the medical image data. In some embodiments, the system may be configured to transmit the generated URL to the second device to allow the second device to display a second view on the second device by replicating the first view. The URL may be used by the second device to generate the second view (e.g., by performing process 300 described herein with reference to FIG. 3). In some embodiments, the system may be configured to transmit the URL in one or more network packets over a communication network (e.g., the Internet). For example, a first clinician at one hospital may want to share the first view of MRI data shown on the system to a second clinician using the second device. The system may transmit the generated URL to the second device to allow the second device to generate a second view of MRI data that replicates visual properties of the first view. In some embodiments, the system may be configured to transmit the generated URL when the system receives user input through a GUI (e.g., by performing process 500 described herein with reference to FIG. 5). In some embodiments, a second view displayed by the second device using the URL may be substantially the same as the first view.

FIG. 3 is a flowchart of an illustrative process 300 for displaying medical images on a device using information received from a different device, according to some embodiments of the technology described herein. Process 300 may be performed by any suitable computing device. For example, process 300 may be performed by user device 106 described herein with reference to FIG. 1.

In some embodiments, process 300 may be performed after a second device (different from the system performing process 300) performs process 200 described herein with reference to FIG. 2. For example, the second device may perform process 200 and transmit the URL to the system performing process 300. Process 300 may begin after receiving the URL from the second device.

Process 300 begins at block 302, where the system obtains a URL indicating parameters associated with a first view of medical image data. The first view of the medical image data may be determined and/or displayed by a second device (e.g., as described at blocks 204-206 of process 200). The system may be configured to obtain the URL indicating the parameters associated with the first view by receiving a transmission of the URL from the second device. For example, the system may receive one or more network data packets through a network (e.g., the Internet) including the URL.

Next, process 300 proceeds to block 304, where the system obtains the medical image data. In some embodiments, the system may be configured to obtain the medical image data from a server storing the medical image data. The system may be configured to obtain a copy of the medical image data from the server. For example, the system may transmit a request to the server identifying the medical image data and receive the medical image data from the server in response to the request.

In some embodiments, the system may be configured to identify the medical image data from the URL. For example, the URL may indicate identifiers of one or more medical image scans (e.g., as shown in FIG. 6A). The system may use the identifiers to obtain the medical image scan(s). For example, the system may use the identifiers of the medical image scan(s) to obtain copies of the medical image scan(s) from a server storing the scan(s). The system may transmit a request including the identifiers to the server to request the scan(s) from the server.

Next, process 300 proceeds to block 306, where the system obtains parameters associated with a second view to be displayed by the system. The parameters may indicate visual properties of the second view of the medical image data. For example, the parameters may include a zoom level, rotation angle relative to an axis, pan relative to a point on an axis, color scheme, contrast, brightness, and/or other parameters.

In some embodiments, the system may be configured to obtain the parameters associated with the second view using the URL. The system may be configured to obtain the parameters associated with the second view by determining the parameters associated with the second view to be the parameters indicted by the URL. For example, the system may determine the parameters by setting the values using user input provided through a GUI. The system may be configured to use the parameters to set visual properties of the second view. For example, the system may set values of variables in a software application (e.g., a rendering software application) using the parameters indicated by the URL. In another example, the system may provide the parameters indicated by the URL as input to one or more functions of the software application. In some embodiments, the system may be configured to provide the URL as input to the software application. The software application may process the URL to identify the indicated parameters and use the parameters to render a display of the second view.

Next, process 300 proceeds to block 308, where the system displays the second view of the medical image data on a display of the system. For example, the system may display the second view on a monitor or touch screen of the system. In some embodiments, the system may be configured to display the second view in a GUI. For example, the GUI may be an Internet website displayed by an Internet browser application. The system may display the second view on the Internet website. In some embodiments, the system may be configured to display the second view of the medical image data by: (1) executing a software application for rendering the second view; and (2) displaying the rendered second view on the system. For example, the system may execute a JAVASCRIPT program using the Internet browser application to render a display of the second view on an Internet website. In some embodiments, the system may be configured to render the second view of the medical image data using ray tracing.

In some embodiments, the user may view the second view shown on the system. In some embodiments, the second view may be substantially the same as the first view. For example, the second view may be a replication of the first view on the system. A clinician using the system may thus examine a view of medical image data (e.g., an MRI scan) that is substantially the same as a view of medical image data being viewed by another clinician using another device (e.g., from which the URL was obtained at block 302).

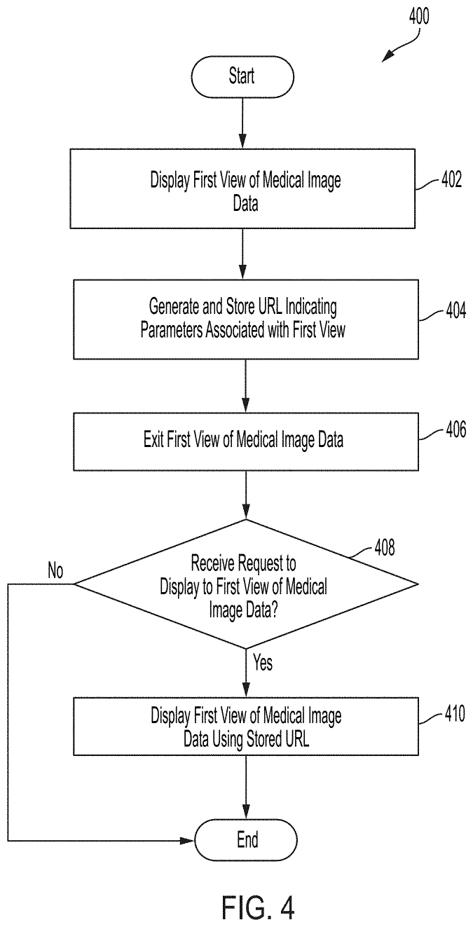

FIG. 4 is a flowchart of an illustrative process 400 for displaying previously viewed medical image data, according to some embodiments of the technology described herein. Process 400 may be performed by any suitable computing device. For example, process 400 may be performed by user device 102 described herein with reference to FIG. 1.

Process 400 begins at block 402, where the system displays a first view of medical image data. The system may be configured to display the first view of the medical image data by: (1) obtaining a copy of the medical image data (e.g., from a medical image data server); and (2) generating a display of the first view by executing a software application (e.g., a JAVASCRIPT program) for rendering the first view. The first view of the medical image data may be associated with parameters. The parameters may indicate visual properties of the first view. For example, the parameters may include zoom, brightness, contrast, rotation angle, and/or other parameters.

In some embodiments, the system may be configured to display the first view in a GUI through which a user can interact with the display of the first view. The system may be configured to allow a user to modify the first view of the medical image data through the GUI. For example, the system may allow the user to rotate an image generated from an MRI scan, zoom in on the image, and/or adjust a brightness of the image.

Next, process 400 proceeds to block 404, where the system generates and stores a URL indicating the parameters associated with the first view. The system may be configured to write alphanumeric characters in the URL to indicate the parameters. For example, the URL may include values for zoom, brightness, and/or contrast of the first view. An example URL indicating parameters associated with a view is described herein with reference to FIGS. 6A-B. The system may be configured to further update the parameters indicated by the URL in response to modifications for the first view. For example, the system may modify a value for zoom in the URL in response to a user modification of the zoom setting in a GUI displaying the first view of the medical image data.

In some embodiments, the system may be configured to automatically store the URL. For example, the system may store the URL in a browsing history of a user. The URL may be stored in a set of URLs indicating different views that a user has viewed. For example, the set of URLs may be a sequence of URLs indicating a sequence of views displayed to the user. In some embodiments, the system may be configured to store the URL when the system receives a user command. For example, the user may request to save the first view (e.g., through a GUI). The system may store the URL in response to the selection. The system may store the URL in association with an identifier for the first view. For example, the system may store the URL in association with a string identifying the first view (e.g., “First View”).

In some embodiments, the system may be configured to store a copy of the medical image data (e.g., obtained to generate the display of the first view). For example, the system may have obtained the copy of the medical image data from a server through a network. The system may store the obtained copy of the medical image data in local storage. For example, the system may store the copy of the medical image data in a local cache. The system may access the medical image data at a later time more quickly than if the system were to obtain another copy of the medical image data from an external server (e.g., over the Internet).

Next, process 400 proceeds to block 406, where the system exits the first view of the medical image data. For example, the system may shut down a software application (e.g., an Internet browser application) used for displaying the first view in response to a user selecting to close the application. In another example, the system may navigate to a different view (e.g., of the same medical image data or other medical image data). The system may exit the first view of the medical image data such that the first view is no longer displayed (e.g., in a GUI for displaying medical images).

Next, process 400 proceeds to block 408. At block 408 the system may receive a request to display the first view of the medical image data. In some embodiments, the system may be configured to receive the request through a GUI. A user may make a selection through the GUI indicating a request to display the first view of the medical image data. For example, the user may make a selection through the GUI to access the first view by clicking on a link associated with the first view (e.g., in a list of links associated with respective views). In another example, the user may select a navigation option (e.g., a back or forward button) to return to a display of the first view from another view.

When the system receives a request to display the first view of medical image data at block 408, process 400 proceeds to block 410, where the system displays the first view of the medical image data using the stored URL. In some embodiments, the system may be configured to: (1) obtain the URL from storage; and (2) use the URL to display the first view. For example, the system may perform process 300 described herein with reference to FIG. 3 to display the first view. The system may use the parameters indicated by the URL to generate the display of the first view. The first view displayed at block 410 may be substantially the same as the first view displayed at 402.

When the system does not receive a request at block 408 to display the first view of the medical image data, then process 400 ends.

FIG. 5 is a flowchart of a process 500 for sharing a view of medical image data, according to some embodiments of the technology described herein. Process 500 may be performed by any suitable computing device. For example, process 500 may be performed by user device 102 described herein with reference to FIG. 1.

In some embodiments, process 500 may be performed as part of process 200. For example, process 500 may be performed at block 212 of process 200. A user may obtain a view of medical image data (e.g., an MRI scan). The user may have obtained the view by making one or more manual modifications (e.g., to a default view). For example, the user may have selected a particular scan from a series of scans, rotated an image, and/or adjusted a brightness of the image. The user (e.g., a first clinician) may want to share the obtained view of the medical image data with another user (e.g., aa second clinician).

Process 500 begins at block 502, where the system generates a GUI element in a user interface for sharing a view. In some embodiments, the system may be configured to generate the GUI element in a GUI of a software application for displaying the view of the medical image data. For example, the system may generate the GUI element in a website displayed in an Internet browser application. In some embodiments, the GUI element may be a button that can be selected (e.g., by clicking, tapping, and/or other user input). For example, the GUI element may be a button labelled “share” or have an icon indicating sharing.

Next, process 500 proceeds to block 504, where the system receives a selection of the GUI element. For example, a user may select the GUI element by clicking and/or tapping the GUI element. In some embodiments, the system may receive a selection of the GUI element in response to an action in the GUI. For example, a swipe across a touch screen of the system may result in the system receiving a selection of the GUI element.

Next, process 500 proceeds to block 506, where the system prompts the user for input specifying a recipient. In some embodiments, the system may be configured to receive an identification of recipient. The identification of the recipient may be a name, a phone number, an email, a username, a group, or other identification of the recipient. In some embodiments, the system may be configured to provide the user with a list of possible recipients. For example, the system may determine a set of contacts associated with the user and provide the set of contacts as options for the user to choose as the recipient.

Next, process 500 proceeds to block 508, where the system transmits a generated URL to a second device for displaying a view of second medical image data on the second device. The generated URL may indicate parameters associated with a view of first medical image data on the system. The first medical image data may be a copy of medical image data obtained by the system (e.g., from a medical image data server). The second device may perform process 300 described herein with reference to FIG. 3 to generate a display of the medical image data. For example, the second device may obtain another copy of the medical image data (e.g., from the server). The copy obtained by the second device may be referred to as second medical image data. The URL, when executed by the second device (e.g., using a software application for displaying views of medical image data), may cause the second device to display a view of the medical image data that is substantially the same as a view of the medical image data shown on a display of the system performing process 500.

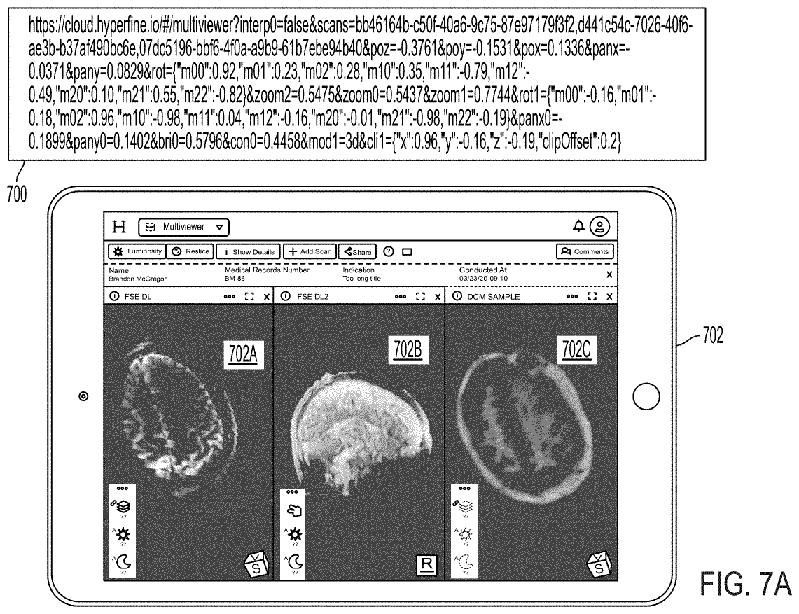

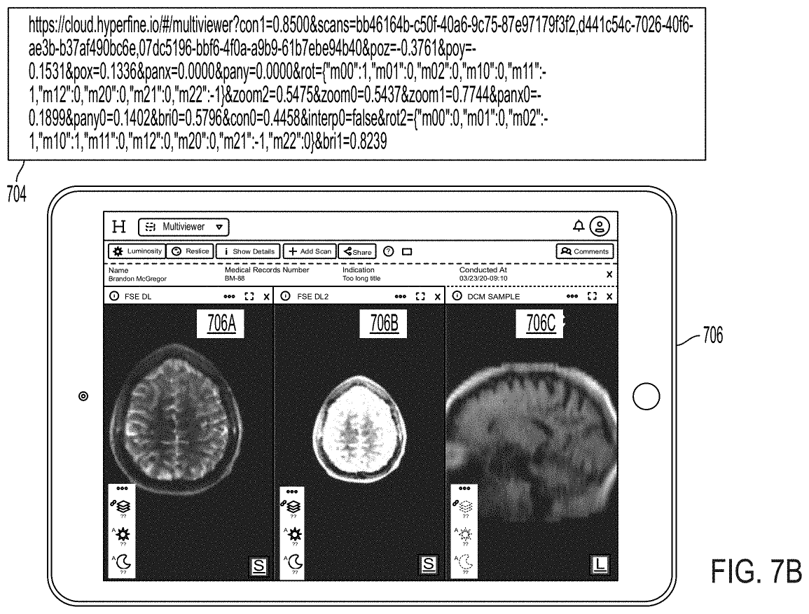

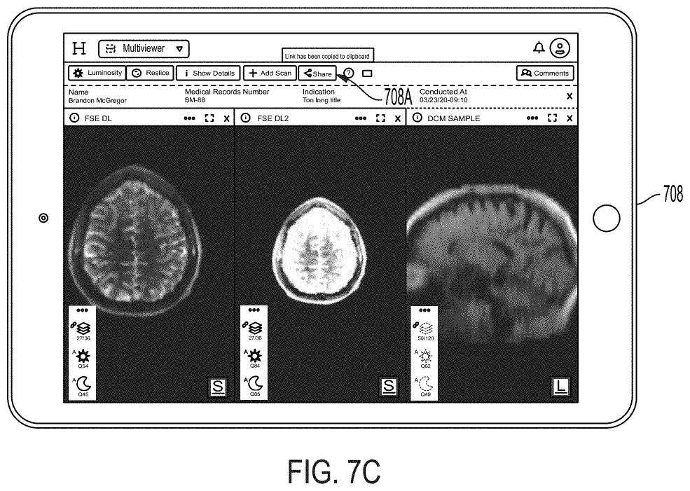

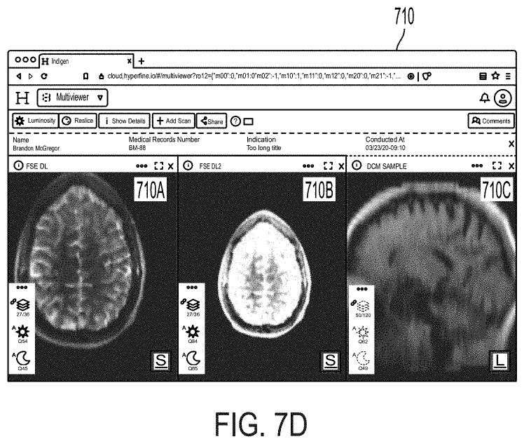

FIG. 7A shows an example of a first URL 700 indicating parameters associated with a first view 702 displayed on a first device. For example, the first URL 700 and the first view 702 may be obtained by performing process 200 described herein with reference to FIG. 2. The first view 702 includes a first pane 702A, a second pane 702B, and a third pane 702C. Each of the panes displays a respective medical image scan. The first URL 700 indicates parameters associated with the first view 702. For example, the first URL 700 includes “poz=−0.3761”, “poy=−0.1531”, and “pox=0.1336” indicating positions of viewing planes in the panes 702A-C. As another example, the first URL 700 includes (1) “rot=“m00”:0.92,“m01”:0.23,“m02”:0.28,“m10”:0.35,“m11”:−0.79,“m12”:−0.49,“m20”:0.10,“m21”:0.55,“m22”:−0.82}” defining a matrix that indicates a rotation for the first pane 702A and the third pane 702C; and (2) “rot1={“m00”:−0.16,“m01”:−0.18,“m02”:0.96,“m10”:−0.98,“m11”:0.04,“m12”:−0.16,“m20”:−0.01,“m21”:−0.98,“m22”:−0.19}” defining a matrix that indicates a rotation for the second pane 702B about an axis. As shown in the example of FIG. 7A, parameters for respective panes are indicated by the first URL 700 using an identifier (e.g., an index number) for the pane. For example, an index number of 0 corresponds to the first pane 702A, an index number of 1 corresponds to the second pane 702B, and an index number of 2 corresponds to the third pane 702C. For example, the first URL 700 includes “zoom0=0.5437”, and “zoom1=0.7744”, and “zoom2=0.5475” indicating a respective zoom for the first pane 702A, the second pane 702B, and the third pane 702C.

FIG. 7B shows an example of a second URL 704 indicating parameters associated with a second view 706 displayed on the first device. The second URL 704 and second view 706 may be obtained as a result of a modification to the first view 702 and/or the first URL 700 (e.g., as described at block 210 of process 200 described herein with reference to FIG. 2). The second view 706 includes a first pane 706A, a second pane 706B, and a third pane 706C. As shown in FIG. 7B, each of the panes 706A-C shows a modified display relative to the panes 702A-C of the first view 702. The second URL 704 indicates parameters associated with the second view 706. For example, the second URL 704 includes “poz=−0.3761”, “poy=−0.1531”, and “pox=0.1336” indicating positions of the viewing planes in the panes 702A-C. The positions of the view planes have not changed from the first view 702. Thus, the first URL 700 and the second URL 704 indicate the same viewing plane positions. As another example, the URL 704, includes (1) “rot=(“m00”:1,“m01”:0,“m02”:0,“m10”:0,“m11”:−1,“m12”:0,“m20”:0,“m21”:0,“m22”:−1}” defining a matrix that indicates rotation for the first pane 706A and the second pane 706B; and (2) “rot2=(“m00”:0,“m01”:0,“m02”:−1,“m10”:1,“m11”:0,“m12”:0,“m20”:0,“m21”:−1,“m22”:0)” defining a matrix that indicates rotation for the third pane 706C. As shown in FIG. 7B, the rotation angle of objects displayed in panes 706A-C of the second view 706 is different from the rotation angle of the objects displayed in panes 702A-C of the first view 702. Accordingly, the rotation parameters indicated by the second URL 704 are different from the rotation parameters indicated by the second URL 700.

FIG. 7C shows an example screenshot 2708 of a user selection of a graphical user interface (GUI) element 708A to share the second view 706 of FIG. 7B shown on the first device. As shown in FIG. 7C, the GUI element 708A is a button labelled “Share” which is selected by a user (e.g., by clicking and/or tapping on a touch screen). In response to selection of the GUI element, the system may allow the user to select a recipient (e.g., as described at block 506 of process 500 described herein with reference to FIG. 5). The system may transmit a generated URL to another device (e.g., as described at block 212 of process 200 described herein with reference to FIG. 2). For example, the system may transmit the second URL 704 in response to a user command (e.g., by selection of the recipient).

FIG. 7D shows a third view 710 shown on a second device different from the first device of FIGS. 7A-C. For example, the third view 710 of FIG. 7D may be obtained by the device by performing process 300 described herein with reference to FIG. 3. The second device may generate the third view 710 of FIG. 7D using the second URL 704 obtained from the first device. The second device may have received a copy of second the URL 704 transmitted by the first device. The second device may use the parameters indicated by the second URL 704 to obtain the third view 710 (e.g., as described in process 300). The third view 710 of FIG. 7D is substantially the same as the second view 706 of FIG. 7B. As shown in the example of FIG. 7D, the second device uses an Internet browser application to render the second view 706 using the second URL 704 (e.g., by executing a JAVASCRIPT program).

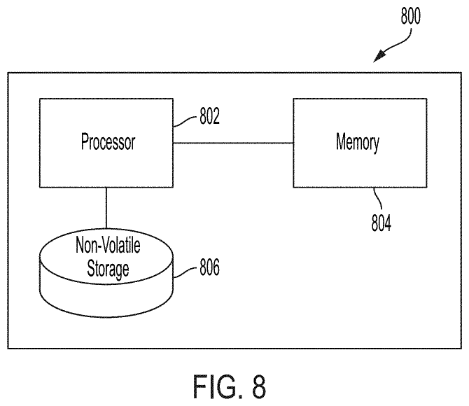

FIG. 8 shows a block diagram of an example computer system 800 that may be used to implement embodiments of the technology described herein. The computing device 800 may include one or more computer hardware processors 802 and non-transitory computer-readable storage media (e.g., memory 804 and one or more non-volatile storage devices 806). The processor(s) 802 may control writing data to and reading data from (1) the memory 804; and (2) the non-volatile storage device(s) 806. To perform any of the functionality described herein, the processor(s) 802 may execute one or more processor-executable instructions stored in one or more non-transitory computer-readable storage media (e.g., the memory 804), which may serve as non-transitory computer-readable storage media storing processor-executable instructions for execution by the processor(s) 802.

The terms “program” or “software” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform tasks or implement abstract data types. Typically, the functionality of the program modules may be combined or distributed.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A method for displaying medical images on a first device by using information received from a second device different from the first device and at least one computer different from the first device and the second device, the method comprising:
using the first device to perform:
obtaining, from the second device via at least one communication network, a uniform resource locator (URL) indicating a first plurality of parameters associated with a first view of medical image data for a subject;
obtaining, from the at least one computer via the at least one communication network, at least some of the medical image data for the subject;
after obtaining the URL and the at least some medical image data for the subject:
displaying a second view of the at least some medical image data on a display of the first device using the first plurality of parameters indicated by the URL.

2. The method of claim 1, wherein the second view is substantially the same as the first view.

3. The method of claim 1, wherein displaying the second view of the at least some medical image data on the display of the first device using the first plurality of parameters indicated by the URL comprises setting a second plurality of parameters associated with the second view equal to the first plurality of parameters.

4. The method of claim 1, wherein displaying the second view of the at least some medical image data on the display of the first device comprises rendering an image using an Internet browser application.

5. The method of claim 1, wherein displaying the second view of the at least some medical image data on the display of the first device comprises executing JAVASCRIPT program that, when executed, causes the first device to render an image from the at least some medical image data on the display of the first device.

6. The method of claim 1, wherein the medical image data comprises magnetic resonance imaging (MRI) data.

7. The method of claim 1, wherein the medical image data comprises ultrasound data, x-ray data, computed tomography (CT) data, or positron emission tomography (PET) data.

8. A system for displaying medical images by using information received from a device different from the system and at least one computer different from the system, the system comprising:
a display;
at least one processor;
at least one non-transitory computer-readable storage medium storing instructions that, when executed by the at least one processor, cause the at least one processor to perform a method comprising:
obtaining, from the device via at least one communication network, a uniform resource locator (URL) indicating a first plurality of parameters associated with a first view of medical image data for a subject;
obtaining, from the at least one computer via the at least one communication network, at least some of the medical image data for the subject;
after obtaining the URL and the at least some medical image data for the subject;
displaying a second view of the at least some medical image data on the display using the first plurality of parameters indicated by the URL.

9. The system of claim 8, wherein the second view is substantially the same as the first view.

10. The system of claim 8, wherein displaying the second view of the at least some medical image data on the display using the first plurality of parameters indicated by the URL comprises setting a second plurality of parameters associated with the second view equal to the first plurality of parameters.

11. The system of claim 8, wherein displaying the second view of the at least some medical image data on the display comprises rendering an image using an Internet browser application.

12. The system of claim 8, wherein displaying the second view of the at least some medical image data on the display comprises executing JAVASCRIPT program that, when executed, causes the at least one processor to render an image from the at least some medical image data on the display.

13. The system of claim 8, wherein the medical image data comprises magnetic resonance imaging (MRI) data.

14. The system of claim 8, wherein the medical image data comprises ultrasound data, x-ray data, computed tomography (CT) data, or positron emission tomography (PET) data.

15. At least one non-transitory computer-readable storage medium storing instructions that, when executed by at least one processor of a first device, cause the at least one processor to perform:
obtaining, from a second device different from the first device via at least one communication network, a uniform resource locator (URL) indicating a first plurality of parameters associated with a first view of medical image data for a subject;
obtaining, from at least one computer different from the first device via the at least one communication network, at least some of the medical image data for a subject; and
after obtaining the URL and the medical image data for the subject:
displaying a second view of the at least some medical image data on a display of the first device using the first plurality of parameters indicated by the URL.

16. The at least one non-transitory computer-readable storage medium of claim 15, wherein the second view is substantially the same as the first view.

17. The at least one non-transitory computer-readable storage medium of claim 15, wherein displaying the second view of the at least some medical image data on the display of the first device using the first plurality of parameters indicated by the URL comprises setting a second plurality of parameters associated with the second view equal to the first plurality of parameters.

18. The at least one non-transitory computer-readable storage medium of claim 15, wherein displaying the second view of the at least some medical image data on the display of the first device comprises rendering an image using an Internet browser application.

19. The at least one non-transitory computer-readable storage medium of claim 15, wherein displaying the second view of the at least some medical image data on the display comprises executing JAVASCRIPT program that, when executed, causes the at least one processor to render an image from the at least some medical image data on the display.

20. The at least one non-transitory computer-readable storage medium of claim 15, wherein the medical image data comprises magnetic resonance imaging (MRI) data.
